(19) United States
(12) Patent Application Publication
(10) Pub. No.: US 2010/0087011 A1
(43) Pub. Date: Apr. 8, 2010

(54) Detection and/or Characterisation of Oligomers

(76) Inventor: Matthew Cooper, Cambridge (GB)

Correspondence Address: FOLEY HOAG, LLP;PATENT GROUP (w/ISA), 155 SEAPORT BLVD., BOSTON, MA, 02210-2600, US

(21) Appl. No.: 12/531,419

(22) PCT Filed: Mar. 17, 2008
(86) PCT No.: PCT/GB08/00947

§ 371 Date: Dec. 24, 2009

(30) Foreign Application Priority Data
Mar. 16, 2007 (GB) 0705088.3

(51) Int. Cl. G01N 33/53; G06F 19/00

(52) U.S. Cl. 436/501; 702/19

(57) ABSTRACT

Disclosed is a method of detecting the presence of an oligomer analyte in a liquid sample, the method comprising the steps of: (a) contacting a sample comprising the oligomer or aggregate with an oscillating sensor surface, which surface may optionally be coated with a receptor that binds directly or indirectly to at least one component of the oligomer or aggregate, so as to cause direct or indirect binding of the oligomer or aggregate to the surface; (b) using a detection circuit to measure or calculate at least two of the following parameters: series resonance frequency (f₀), and hence the series resonance frequency shift (ΔF); motional resistance (R_{M}), and hence the motional resistance shift ΔR; motional inductance (L_{M}); motional capacitance (C_{M}); parallel capacitance (C₀); frequency half-band half-width (Γ); Q-factor (=f/(2Γ); dissipative factor (1/Q); impedance or admittance phase (φ) and hence phase shift (Δφ); and impedance or admittance amplitude (Z, or Y) and hence amplitude change (ΔZ, or ΔY); (c) and analysing the calculated values to derive data which vary according to the presence and/or amount of oligomer in the sample; (d) and, optionally, repeating the measurements continuously or intermittently to derive data which vary according to the presence and/or amount of oligomer or aggregate in the sample to be calculated as a function of elapsed time.

### FIELD OF THE INVENTION

The invention relates to a method of, and apparatus for, detecting and/or characterising an oligomer analyte in a sample.

### BACKGROUND TO THE INVENTION

Many biotechnological processes are based on specific properties, such as the binding affinities, of one or more biological or chemical entities. For example, separation techniques may aim to separate one or more different entities having specific properties from a sample. A biosensor or analytical method may aim to detect only chemical or biological entities having specific properties, which may be present in a sample.

In such processes, it is often important to discriminate between entities with similar properties. For example, a separation technique, such as affinity chromatography, or a biosensor, may need to discriminate between similar entities with only subtle differences therebetween. Examples of properties which can be used to discriminate between biological or chemical entities include their size, mass, isoelectric point, presence or absence of labels, composition, structure, or the presence of recognition sites to which specific binding means can bind.

Biotechnological processes can be made to be specific by including process steps that use binding means with specific affinity for biological or chemical entities. Example binding means include antibodies or antibody fragments, chemical ligands, or nucleic acid sequences which have affinity for chemical and biological entities including specific recognition sites to which binding means bind. For example, antibodies or antibody fragments bind to regions of their ligands referred to as epitopes.

Chemical or biological entities which are similar to each other in many ways but which have different recognition sites can be discriminated between if binding means with different affinities for the different recognition sites can be found. However, it is not possible to discriminate between entities in this way if they have only similar or identical recognition sites, or if binding means able to discriminate with sufficient specificity between two similar entities cannot be found or are not commercially viable.

Thus, antibodies, and other binding means for binding specific recognition sites often cannot discriminate between an entity which is present as a plurality of unlinked monomers in a sample, and an entity which is present in the form of an oligomer comprising a plurality of monomers. This is because the molecule may have the same or similar binding properties (such as presenting the same or similar recognition sites to specific binding means) whether it is present as a monomer or an oligomer.

Processes which are capable of discriminating between chemically similar entities on the basis of their oligomerisation are useful in fields including biochemistry, biotechnology, microbiology, polymer-science and materials science.

There is a great deal of interest in detecting diseases of the brain, in animals or humans in which proteins form aggregates within cells in afflicted individuals. For example, Alzheimer's Disease is characterised by the formation of aggregates of β-amyloid peptide. Such aggregates typically comprise proteins which have a normal, non-disease state form present in the cells of the central nervous system as discrete non-aggregated monomers; and also have a disease-state form in which they can aggregate. The disease and non-disease state forms may differ only in terms of configuration, and/or may have chemical differences. It is therefore desirable to determine whether such proteins are present in oligomerised or discrete non-oligomerised (monomer) form. A discrete protein and an oligomer of many proteins will both have similar properties and the same, or similar, epitopes to which antibodies and other binding means can bind, and so are hard to discriminate between by virtue only of the affinity with which they are bound by antibodies.

Some diseases in which proteins form disorganised oligomers in the cells of sufferers are believed to be transmitted by proteinaceous infectious particles referred to as prions which are typically modified forms of mammalian proteins. It is desirable to detect these aggregated protein oligomers and to enable the protein oligomers to be discriminated from non-oligomorised (or less oligomerised) prion proteins, whether in their non-disease state form or modified disease-state form. In some diseases the infectious particle is thought to be an aggregate of oligomerised proteins and it is desirable to detect this infectious oligomer.

In some applications, an entity which is present in a sample will be detected or measured. Immunological techniques, such as sandwich immunoassays, are known which can quantify the number of recognition sites present in a sample. However, they cannot in general discriminate between whether these recognition sites are present within an oligomer or whether those recognition sites are present on individual components.

Oligomers comprising a plurality of molecular components could in principle be discriminated from non-oligomerised or less-oligomerised components by virtue of their different masses. However, conventional mass-sensing methods used in biotechnology, such as mass spectrometry, surface plasmon resonance detection, the use of field effect transistors, or enzyme linked immunosorbent assays (ELISAs), cannot discriminate between, for example, a) one oligomer of one hundred thousand monomer sub units, and b) one hundred thousand discrete monomer sub units. In general, both a) and b) will have identical or similar masses, and will often also present similar, or identical, recognition sites to binding means in similar or identical numbers.

Therefore some embodiments of the present invention aim to detect or quantify the presence of oligomerised components, perhaps in the presence of non-oligomerised discrete molecular components, which non-oligomerised molecules may be present in greater concentration than the oligomer.

The invention is especially relevant to the detection of diseases such as Alzheimer's Disease, Parkinson's Disease, Huntington's Disease, Creutzfeld-Jakob Disease, new variant Creutzfeld-Jakob Disease, Gerstmann-Sträussler-Scheinker Disease, Kuru, Bovine spongiform encephalopathy and fatal familial insomnia.

Alzheimer's Disease is associated with the 4-4.5 kDa, 39-43 residue β-amyloid (βA4) peptide. This peptide is protolytically cleaved from three larger proteins encoded by alternative splicing of the β-amyloid protein precursor gene (βAPP). βAPP proteins are usually O- and N-linked glycosylated transmembrane proteins of 695, 751 and 770 residues with a 47 residue cytoplasmic domain. βA4 corresponds to 28 extracellular residues and 15-16 transmembrane residues of the βAPP proteins. Proteolytic processing of βAPP proteins at a position equivalent to residue 16 of βA4 usually leads to shedding of the extracellular domain. However, an inappropriate cleavage event leads to generation of soluble, cytoplasmic βA4. Fibrillar oligomers of βA4 are formed when the protein is transformed by partial denaturing to the β-sheet configuration, but only very slowly at physiological pH of 7-7.5. The oligomerisation process is more rapid at the lower pH of 5-6 (as found in some sub-cellular compartments), but also requires additional factors such as radical generation or metal-catalysed oxidation systems. It is the oligomerisation of βA4 into fibrillar bundles that ultimately leads to neuronal cell death and the onset of dementia.

Accordingly it is desirable to detect βA4 aggregates, perhaps in the presence of either or both discrete unaggregated βA4 or correctly processed βAPP proteins.

Parkinson's Disease is associated with alpha-synuclein, a 14 kDa protein which in non-disease-state form is an intrinsically unstructured/unfolded presynaptic protein. However, when it is oxidised at tyrosine or methionine residues, it enters a partially folded, disease-state form which accelerates its polymerisation to form amyloid-like fibrils. In Parkinson's Disease, these fibrils lead to degeneration of dopaminergic neurons of the substantia nigra and Parkinsonia motor deficits. There is genetic evidence for a direct role of alpha-synuclein in early onset, familial Parkinson's Disease, including mutations (G209A) that enhance its stability and propensity to cause fibrils. Accordingly it is desirable to detect the formation of oligomerised alpha-synuclein (in polymerised form), perhaps in the presence of discrete alpha-synuclein proteins (whether unoxidised or oxidised, normal or mutant).

The causative agent of neurodegenerative Huntington's disease is the ubiquitously expressed 55-60 kDa huntingtin protein. The huntingtin protein has little homology to other proteins, but in the disease state is characterised by amplification of a CAG codon in the open reading frame, leading to glutamine repeats in the mature protein. This poly-glutamine region (comprising perhaps 80-100 glutamine repeats) in the full length protein leads to cytoplasmic aggregation, while smaller N-terminal poly-glutamine-rich fragments can form nuclear oligomers, resulting in neuronal death. It is therefore desirable to detect oligomers of the full length protein or N-terminal fragments, perhaps in the presence of protein/protein fragments with fewer or no glutamine components.

In Creutzfeld-Jakob disease and Gerstmann-Straussler-Scheinker disease, the monomer prion protein exists in a ubiquitously-expressed, normal non-disease state cellular form (PrP^{c}), and a refolded, protease resistant, heat resistant, infectious disease-state form. For example, in the disease-state form of ovine transmissible spongiform encephalopathy neurodegenerative disease (scrapie) is referred to a PrP^{Sc}. PrP^{Sc} is able to convert cellular PrP^{c} to the infectious disease-state PrP^{Sc} form. It is believed that the protease resistance of PrP^{Sc} (and mutant forms of PrP^{c}, e.g. P105L, D178N-129N, T183A, F198S) leads to the sequestration of the protein in inclusion bodies, where it self-assembles into beta-sheet oligomers, ultimately forming fibrils characteristic of the neurodegenerative Creutzfeld-Jakob Disease, Gerstmann-Straussler-Scheinker Syndrome and fatal familial insomnia.

It is therefore desirable to detect oligomers of disease-state forms of PrP^{c} and other prion proteins, perhaps in the presence of non-oligomerised disease-state or non-disease-state forms of PrP^{c} and other prion proteins.

It is known that piezo-electric and acoustic transducer-based biosensors can be used to detect small increases in mass caused, for example, by binding of an analyte in a sample to a receptor molecule immobilised on the surface of the transducer, the receptor having specific binding activity for the analyte.

Such biosensors operate by causing the transducer surface to resonate, under the control of a driver. Signals from the sensor are received and processed at a receiver. The driver and receiver may, with the sensor, form part of an oscillator circuit with positive feedback so that the sensor is made to oscillate at the resonant frequency of the circuit, which frequency is related to the mechanical resonant frequency of the sensor. This in turn depends on a number of factors, including its mass. Thus binding of an analyte to the transducer surface causes an apparent increase in mass, which manifests itself by a shift in the resonant frequency of the sensor and hence of the circuit.

Alternatively the driver and receiver may form part of a network analyser which oscillates the sensor surface at a frequency which is swept through a range of frequencies, including the resonant frequency, and which analyses the frequency-dependent impedance or admittance of the sensor over that range. At the series resonant frequency there is a dip in the impedance.

The relationship between resonant frequency and deposited mass is described by the Sauerbrey equation, but this is only valid for thin, solid layers deposited on the oscillating surface, surrounded by a gas or a vacuum. In practice, most analytes are contacted with the sensor surface in a liquid, the properties of which (especially its density and viscosity) will affect the behaviour of the transducer. Additionally, most analytes will not behave as perfectly rigid bodies. These factors cause a dampening of the oscillations, which can be modelled as a resistance in the equivalent electrical circuit, according to the standard Butterworth/Van Dyke model, which models the behaviour of the mechanical oscillator as an equivalent electrical circuit comprising a capacitance, an inductance and a resistance in series, all in parallel with another capacitance.

WO 01/23892 (Sensorchem International Corporation) describes how the properties of an oscillating sensor will depend on, inter alia, the degree of “coupling” between the oscillating surface and a surrounding viscous liquid, and defines a “slip parameter”, α, to describe the efficiency of this coupling. In particular the document discloses “a process for sensing biological or chemical changes in molecular structural shape or mass of molecules attached to the surface of an . . . oscillating molecular sensing device”. The process comprises, inter alia, collecting data to determine values for each of the following parameters: series resonance frequency shift (Fₛ), motional resistance (R_{M}), motional inductance (L_{M}), motional capacitance (C_{M}), electrostatic capacitance (C_{O}) and boundary layer slip parameter (α). However, the document is very brief and does not clearly teach how changes in the measured parameters can be qualitatively related to changes in molecular structural shape.

WO 99/30159 (Q-Sense) discloses the use of ΔF vs. ΔR_{M} plots to display the binding data from a TSM sensor, and that these show typically linear behaviour, and can have different slopes, which can be used to distinguish different binding of a common antibody to different epitopes immobilised on the surface of the sensor having the same affinity for the antibody. The different slopes of the plots are attributed to different conformational states (tertiary structure). It does not disclose any method for the analysis of oligomerisation (quaternary structure), nor does it disclose the use of R_{M} or any other parameter in the analysis of conformational state.

WO 04/040317 (Farfield) discloses that the extent of oligomerisation of proteins, and specifically Aβ amyloids can be determined by using a sensor. An antibody to the aggregate or amyloid is immobilised on the surface and exposed to the aggregate in a liquid medium. The sensor responds to a change in “the localised environment rendered by” the presence of the aggregate. The invention is disclosed as “based on the recognition that exploiting a sensor device sensitive to mass changing events at the molecular level to detect the extent of aggregation of a protein to which it is exposed may be a useful ex vivo (or even potentially in vivo) diagnostic tool . . . ”. The response may be due to other physical parameters that change as a result of binding including (inter alia) a visco-elastic property, and the sensor may be (inter alia) an acoustic sensor. However the application does not disclose how, in the case of an acoustic device, any specific property can be related to the extent of aggregation.

WO 2006/063437 (University of Guelph) discloses that measurement of F and R_{M} made using a TSM sensor can be used to detect binding of PrP^{Sc} in a sample to immobilised PrP^{C}. Binding of the hydrophobic PrP^{Sc} to the hydrophilic PrP^{C} (or alternately conversion of the PrP^{C} to PrP^{Sc}) may cause changes in R_{M}, and hence F and R_{M} may vary at different rates during the interaction. The time course of the response may reveal the presence of PrP^{Sc}. However only analysis based on measurements of F(t) curves are disclosed. As it is known in the prior art that R_{M} and F, and indeed the other parameters that may be measured, are not correlated during binding, this application only shows that it is possible to identify mass sensitive behaviour characterising the presence or interaction of the two proteins.

None of the above prior art clearly discloses a method involving measurement of two parameters to determine quaternary structure or oligomerisation processes on surfaces.

### SUMMARY OF THE INVENTION

The present invention provides a method of detecting the presence of an oligomer analyte in a sample, especially in a complex sample that may comprise a mixture of oligomer and non-oligomerised molecules. The invention also provides a method for characterising the oligomerisation of an analyte molecule. In a particular embodiment the invention provides a method for determining the extent or degree of oligomerisation of an analyte.

For present purposes, an oligomer may be defined as a complex that is comprised of two or more molecular subunits. (The subunits could be monomers or could themselves conceivably be oligomers). An oligomer may be a homo-oligomer (in which the two or more subunits are identical to one another) or may be a hetero-oligomer (in which these are at least two different subunits e.g. A-B-A-B-A-B- etc.). The subunits present in an oligomer may be held together by any one or more of van de Waal's forces, covalent bonds, non-covalent bonds, hydrophobic interactions, hydrogen bonds and the like.

The linkage between subunits in the oligomer may be regular and systematic, or may be irregular and disorganised. An irregular and disorganised oligomer may sometimes be referred to as an “aggregate”, and an aggregate, for present purposes, is simply one type of oligomer. The order of oligomerisation (that is, the number of subunits in the oligomer) may be any number from 2 or above. Typically the number of subunits will be between 2 and 10,000, more typically between 2 and 1,000, frequently between 2 and 100.

Generally, especially in biological samples, the subunits will comprise a polypeptide chain. For present purposes, a polypeptide chain is defined as a continuous chain of ten or more amino acid residues, adjacent residues being joined by a peptide bond. The polypeptide may additionally contain other moieties, such as sugars, fatty acids, phosphate groups and the like, all of which are known to be present in some naturally occurring polypeptides.

Typically, but not necessarily, oligomers will be water-soluble with a solubility of at least 100 mg/L, and will comprise correctly-folded subunits. Such complexes are abundant in nature and knowledge of the oligomeric state (i.e. dimer, trimer, quatromer, etc.) can be important in the study of the function of the oligomer, or in the discovery of drugs or diagnostic tests associated with the oligomer.

However, as described above, there are a number of disease states that are associated with abnormal oligomerisation. An abnormal oligomer will typically comprise one or more mis-folded subunits (i.e. the subunit will have a tertiary structure which is different to that found in oligomers not associated with the disease state) and will typically be insoluble or poorly soluble in water i.e. with a solubility of less than 100 mg/L. The present method can be used detect soluble oligomers and to detect insoluble oligomers (such as β-amyloid fibrillar complexes).

Further, by way of explanation, an oligomeric protein complex is considered to have four levels of structure. The primary structure is the sequence of amino acid residues in the polypeptide chains that make up the complex. The secondary structure is the presence and arrangement of structural motifs in the chains i.e. α-helix, β-sheet, beta barrel, WD40 motifs etc. The tertiary structure is the overall three-dimensional conformation of the separate chains. The quaternary structure relates to the number and arrangement of the chains in the oligomeric complex.

The terms “oligomerised” and “non-oligomerised” are used in the present specification. The terms are not intended to be interpreted as absolute. That is, it is possible that an “oligomerised” composition may (and normally will) comprise at least some non-oligomerised subunits, and equally that a non-oligomerised composition will frequently contain at least some oligomerised subunits.

Thus, typically an oligomerised composition is one which is “substantially oligomerised”. A substantially oligomerised composition is one in which at least 51% of the mass of oligomerisable subunit of interest in the composition is present as an oligomer, or at least 60%, or at least 65%, or at least 70%, or at least 75%, preferably at least 80%, more preferably at least 85%, most preferably at least 90%.

Equally, a non-oligomerised composition is typically one which is “substantially non-oligomerised”. A substantially non-oligomerised composition is one in which at least 51% of the mass of oligomerisable subunit of interest in the composition is present as discrete subunits, or at least 60%, or at least 65%, or at least 70%, or at least 75%, preferably at least 80%, more preferably at least 85%, most preferably at least 90%.

The oligomer analyte may be present in the sample initially. Alternatively, the method may be performed by, for example, contacting the sensor surface with a sample comprising plurality of non-oligomerised subunits and monitoring or detecting formation of the oligomer from the subunits. The oligomerisation reaction can, for instance, be triggered or facilitated by altering the assay conditions and/or adding a catalyst to catalyse the oligomerisation.

The invention also provides apparatus for use in performing the method of invention.

More specifically, the invention provides a method of detecting the presence and/or amount of an oligomer analyte in a sample, the method comprising the steps of:
(a) contacting a sample comprising the oligomer with an oscillating sensor surface, which surface may optionally be provided with a receptor that binds directly or indirectly to at least one component of the oligomer, so as to cause direct or indirect binding of the oligomer to the surface;
(b) using a detection circuit to measure or calculate at least two of the following parameters:
   series resonance frequency (fₒ), and hence the series resonance frequency shift (ΔF);
   motional resistance (R_{M}), and hence the motional resistance shift ΔR;
   motional inductance (L_{M});
   motional capacitance (C_{M});
   parallel capacitance (C₀);
   frequency half-band half-width (Γ);
   Q-factor (=f/(2Γ);
   dissipative factor (1/Q);
   impedance or admittance phase (φ) and hence phase shift (Δφ); and impedance or admittance amplitude (Z, or Y) and hence amplitude change (ΔZ, or ΔY);
(c) and analysing the calculated values to derive data which vary according to the presence and/or amount of oligomer in the sample;
(d) and, optionally, repeating the measurements continuously or intermittently to derive data which vary according to the presence and/or amount of oligomer in the sample to be calculated as a function of elapsed time.

The method of the invention can not only distinguish between the binding of oligomers and monomers or other subunits to the sensor surface, but can also distinguish between different samples in which there are relatively large amounts of oligomer, and those in which there are relatively small amounts of oligomer. Accordingly, in a second aspect the invention provides a method of determining, in relative terms, the amount of oligomerised substance (especially polypeptide) present in a sample.

This could have particular significance in situations where the amount of oligomerised protein present in a sample has prognostic significance. For example, the state of progression of Alzheimer's disease, in terms of clinical symptoms, may be related to the relative amount of aggregated β-amyloid peptide present in samples.

In some circumstances the invention may allow for the determination of a ratio of oligomer subunit. In particular, this could be achieved if there is prior knowledge of the response of the sensor to subunit alone. Deviations from this behaviour calculated from at least two of the parameters listed above can then allow determination of the ratio of oligomer:subunit based on the degree of deviation from the response due to subunit alone.

In some embodiments, the methods of the invention may comprise determination of the amount of subunit (or monomer) in the sample, which would allow an accurate measurement of the ratio of oligomer:subunit in the sample. The amount of subunit could be quantified either using an oscillating sensor surface as in the methods described above, or may lend itself to more conventional quantification techniques such as ELISA or the like. The sample could be treated (e.g. by precipitation, filtration or ultracentrifugation) to remove or separate oligomer from non-oligomerised subunits.

In other embodiments, the relative or absolute amount of molecules with a particular order of oligomerisation could be determined. For example, comparison of experimental results by reference to a known standard would allow such a calculation to be made. Accordingly, in some embodiments, the method of the invention may be performed to calculate the relative or absolute amounts of molecules with different orders of oligomerisation. This may be especially significant in some diseases, where a disease state is more strongly associated with a particular oligomer. (e.g. Alzheimer's is thought possibly to be most strongly associated with a trimeric complex).

References herein to “extent of oligomerisation” are intended to encompass both ratios of oligomer:subunit; and order of oligomerisation.

The oscillating sensor surface may be part of a piezo-electric device, a surface transverse shear mode (TSM) device such as quartz crystal resonator, a surface acoustic wave (SAW) device, an acoustic plate mode device, a flexural plate wave device, a magnetic acoustic device, an atomic force microscopy device, a micro-electromechanical device, or even a tuning fork or derivatised membrane. Piezo-electric, shear mode devices are preferred. Particularly preferred are quartz crystal resonators.

It is well known to those skilled in the art how to construct and use oscillating sensor surfaces such as TSM devices and associated components (such as network analysers). The method of the invention will typically comprise use of an essentially conventional sensor. Suitable teaching and guidance is given, for example, in the following: WO 00/26636; WO 02/12873; U.S. Pat. No. 6,589,727; U.S. Pat. No. 6,647,764; WO 2004/095011; WO 2004/095012; WO 2005/121769; and WO 2006/027582.

The sensor may conveniently comprise any conventional piezo-electric or piezo-magnetic material, such as quartz, lithium tantalate, lithium niobate, gallium arsenide, aluminium nitride, zinc oxide, polyvinylidene fluoride and the like, although quartz is generally preferred.

Typically the sensor does not bind directly to the analyte. Instead, there is immobilised on the sensor one or more types of receptor molecule which bind to the analyte, preferably in a specific binding reaction. “Specific binding” as used herein, means that the receptor has a relatively high binding affinity only for the analyte molecule or molecules which comprise at least a portion which has considerable structural similarity with the analyte, and relatively low binding affinity for dissimilar molecules. Typically the binding affinity for the analyte will be such that the K_{d} value will be 10 μM or lower, preferably lower than 1 μM, more preferably lower than 100 nM, or even lower than 10 nM.

Conventionally the sensor surface is coated with a thin layer of an electrically conducting substance, such as gold, platinum, silver or copper, which acts as an electrode. Preferably, but not necessarily, the electrode surface is provided with a coating, desirably hydrophilic (to allow wetting by aqueous samples), which is resistant to non-specific binding of analytes and contaminants. Suitable such coatings include dextran, hyaluronic acid, sepharose, agarose, nitrocellulose, PVA and PMMA (polymethyl methacrylate). The electrode may also support an adhesion layer, intermediate between the electrode and the polymer coating. This adhesion layer may comprise a thiol-anchored self-assembled monolayer (SAM) or a silane-coupled layer. Alternately the analyte may be bound directly to the adhesion layer after activation.

Ultimately, there will generally be attached either directly, or more preferably indirectly, to the electrode surface one or more types of receptor molecule that will bind to the analyte(s) of interest. The receptor molecule will most conveniently comprise an antibody or an antigen-binding fragment or variant of an antibody, such as F_{V}, single chain F_{V}, Fₐb¹, F(ab¹)² a single domain antibody, or an oligomer of any of these. Alternatively, depending on the nature of the analyte, the receptor molecule may comprise an antigen, a peptide (e.g. a substrate or substrate analogue), a nucleic acid-binding protein, an enzyme, a single stranded oligo- or polynucleotide, a molecule containing at least a ligand-binding portion of a eukaryotic cell surface receptor (e.g. a hormone receptor), a lectin, a polysaccharide, an aptamer, an affibody, an aggregate-specific polymer and so on. Again, suitable methods for making appropriate sensor surfaces are known to those skilled in the art and taught in, for example, U.S. Pat. No. 5,436,161, U.S. Pat. No. 5,242,828, U.S. Pat. No. 5,763,191 and EP 0,515,615.

The receptor molecules may be essentially randomly distributed across the surface of the sensor, or may be concentrated at spatially distinct sites, especially at loci which may serve as nucleation sites for oligomerisation of the analyte e.g. around a protease tethered to the sensor, which protease has an activity which promotes or inhibits oligomerisation.

The sample is typically a liquid sample. In particular, the sample may comprise a sample of body fluid from a subject, such as blood, serum, plasma, interstitial fluid, breath condensates, ocular lens liquid, lachrymal fluid, urine, milk, mucus, sputum, synovial fluid, peritoneal fluid, transdermal exudates, pharyngeal exudates, bronchoalveolar lavage, tracheal aspirations, semen, vaginal or urethral secretions saliva, cerebrospinal fluid or the like. Alternatively the sample may comprise an extract or lysate from e.g. a biopsy (e.g. neuronal tissue), a tissue culture, a phage or other antibody display library etc. The liquid may comprise an organic solvent, but will normally be essentially aqueous.

The method of the invention is such that the two or more parameters are calculated for the same sample. In practice, a sample (especially a biological sample) is usually sub-divided into a number of aliquots, to allow for repeat measurements etc. The present invention is such that the two or more parameters are preferably calculated for the same aliquot, where the sample is so sub-divided. Indeed, in preferred embodiments, the two or more parameters are generally calculated or derived from data collected during the same assay or experiment run.

The analyte may be a dimer, trimer, or higher order multimer or any mixture or combination thereof. The analyte may be an antigen, peptide or other small molecule, a polypeptide, a steroid, a protein, a prion, a eukaryotic cell, a bacterium, a virus or other micro-organism (e.g. a yeast, a protozoon, a chlamydia or Rickettsia) and the like. Typically the sample may comprise both oligomers (or aggregates) and subunits or monomers thereof. Often the sample will comprise an excess (in numerical terms) of subunit compared to oligomer, which means that any test must be very sensitive in order to detect the few molecules of oligomer that may be present.

Most especially, the sample may be a sample of clinical significance, in which there may be, for example, aggregated β-amyloid protein or peptides, mis-folded prion protein aggregates, or aggregates of Huntingtin protein.

The person skilled in the art will appreciate that the parameters defined above are not all independent. Thus, for example, the dissipative factor, D, is the reciprocal of the Q-factor; the impedance and admittance phase and amplitude are all dependent on R, L and C values.

In practice the two or more parameters may advantageously be determined or calculated by measuring the admittance of the sensor (or its impedance, the inverse of admittance) over a range of frequencies. At the resonance frequency there is a peak in admittance (and a trough in impedance), and by measuring the admittance or impedance values over a range of frequencies around the resonance it is possible to determine the resonance frequency, by fitting the array of measured values to an to an equation derived from a mathematical model describing the behaviour of the sensor as an equivalent circuit or other physical model. A number of models have been developed, which incorporate different levels of approximation, corresponding to the interaction between the mass dependent and viscosity-density dependencies. A commonly used model is the Modified Butterworth Van Dyke equivalent circuit, but transmission line type models have also been used.

Other variants of this are known in the art; for example because the impedance (and admittance) are complex numbers mathematically, they have a real and imaginary part (or alternately a phase and amplitude). It is also known to measure only one component of the complex value, and to fit to equations describing the behaviour of this single component only.

By comparing the resonance frequency of the unloaded sensor (typically, a reference sensor as closely similar as possible to the measurement sensor) with the loaded sensor during and/or after contact with the sample(s), it is possible to calculate the change in resonance frequency, ΔF. The reference sensor may comprise a coating of a biological material which is inert to the analyte.

The motional resistance R_{M} of the loaded sensor can be directly determined by the same fitting method. Equally, the change in motional resistance, ΔR_{M}, between the R_{M} values of the unloaded sensor and the sensor exposed to sample, can be readily calculated.

The applicants have found that analysis of the relationship between ΔF and ΔR_{M}, (henceforward called just ΔR when describing experimental results) is especially helpful in revealing information concerning the conformation and/or degree of oligomerisation or aggregation of an analyte. Examples of such analysis include: preparing plots of ΔR against ΔF (or vice versa) and determining the gradient and/or degree of curvature of the resulting plot; plotting 1/ΔR against 1/ΔF (or vice versa) and calculating or estimating the displacement of the data points from the x and/or y axes. A further example of such analysis is to plot ln(absolute value of ΔR/ΔF) against time for analyte association and/or dissociation from the sensor.

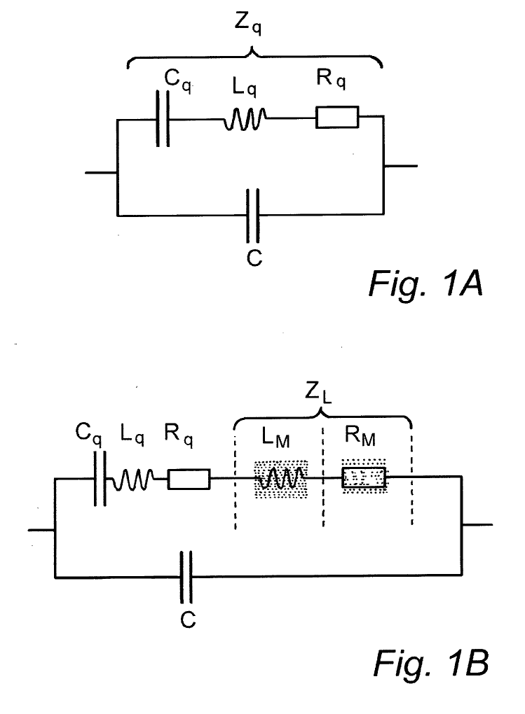

Depending on the model adopted, there may be different parameters which can be measured. The applicants prefer to use the modified Butterworth Van Dyke model (see FIG. 1), in which the inductance (L_{q}), resistance (R_{q}) and parallel holder capacitance (C₀) of the oscillating quartz transducer are augmented by a complex (i.e. multi-component) series impedance Z_{L}, attributed to the load on the transducer arising from the mass of material attached to the sensing surface, and the physical properties thereof. The load impedance is conventionally considered to consist of a resistive component, the load resistance (R_{L}), and a reactive component (X_{L}). X_{L} is in turn decomposed into a capacitive and inductive component (C_{L}) and (L_{L}).

The modified Butterworth Van Dyke (MBVD) model has the virtue of relative simplicity whilst giving reasonably reproducible data. In the MBVD model there are only 5 parameters which may be varied to achieve a fit of obtained admittance data, as a function of frequency, to the model equation. These 5 parameters are:

1. series resonance frequency (the frequency of maximum admittance) (fₒ);
2. the motional capacitance C_{M};
3. the motional resistance R_{M};
4. the motional inductance L_{M}; and
5. the parallel capacitance C₀

Referring to FIG. 1, where R_{q1}, L_{q} and C_{q} all refer to motional parameters of the unloaded sensor, in all realistic cases the components C_{L} and C_{q} are in series and thus: 1/C_{M}=1/C_{q}+1/C_{L}. Because C_{q}<<C_{L}, C_{L} may be ignored in the analysis to leave Y_{M} dependent only on R_{L} and L_{L} and C_{q}.

It can further be seen that

R_{M}=R_{q}+R_{L}, and; L_{L}=L_{q}+L_{L}. Because R_{q}<<R_{L}, and L_{q}<<L_{L}, the significant parameters reduce to the five listed above: f₀, C_{q}, R_{L}, L_{L}, C₀.

Accordingly, the methods of the invention preferably involve calculation or derivation of at least two of the 5 parameters defined above. It will be appreciated that within the model there exists further choices of parameter sets, which are mathematically linked to the five conventional parameters. For example Q (=1/2fR_{M}C_{M}), or the wave resistance (ρ_{w}=1/2fC_{M}) may be chosen, as a matter of convenience for the chosen fitting algorithm.

In general terms, the method of the invention involves measurement of two or more parameters, which are related to (a) the mass of substance attached (directly or indirectly) to the sensor, and (b) the viscoelastic properties of this mass. Changes in the oligomerisation of the analyte cause changes in the viscoelastic properties (and possibly also the mass distribution) of the attached mass, generating a detectable change in the measured properties of the sensor that can be related, in a manner described below, to changes in the oligomerisation of the analyte.

In preferred embodiments, the invention involves calculation of between two and five (inclusive) of the parameters previously recited in step (b). In a more preferred embodiment, the invention involves calculation of between two and four (inclusive) of the parameters previously recited in step (b). In an even more preferred embodiment the invention involves calculation of two or three of the parameters previously recited in step (b). The most preferred embodiment of the invention involves calculation of just two of the parameters.

In a preferred embodiment the invention comprises calculation of the series resonance frequency, and hence the resonance frequency shift, ΔF.

In a preferred embodiment the invention comprises calculation of the motional resistance R_{M}, and hence the motional resistance shift, ΔR_{M}. In a particularly preferred embodiment the invention comprises calculation of both ΔF and ΔR_{M}.

The inventors have found that analysis of ΔF and ΔR can give useful information as to the presence or absence of oligomers, and to the relative amount or extent of oligomerisation. Data relating to ΔF and ΔR can be analysed in any of several different methods, which will be apparent to those skilled in the art with the benefit of the present disclosure. In general terms, comparison of ΔF with ΔR and/or their derivatives dF/dt with dR/dt, gives useful information.

In general terms, the inventors have found that, during the course of capture of a globular non-oligomerised protein molecules, the change of frequency and change of resistance, ΔF and ΔR respectively, has a constant ratio, so that plots of ΔR against ΔF (or ΔF against ΔR) with assay time as the parameter are linear. Moreover, this is true regardless of the molecular weight of the protein (e.g. this has been experimentally determined with ubiquitin, MW 8,500 Da; Ovalbumin, MW 45 kDa; BSA, MW 66.4 kDa; and IgG, MW 150 kDa).

The gradient of the linear ΔR vs. ΔF plot does vary slightly from protein to protein. In the sample soluble, globular protein analytes so far investigated, the gradient varies from 0.014 to 0.021, with an average value of about 0.017 Ohm.seconds. This linear relationship for ΔF and ΔR has also been observed by the inventors for hybridisation of short chain 24 mer ssDNA hybridisation to an immobilised 24 mer ss DNA probe (i.e. it is not limited to capture of protein molecules).

The inventors have found that, in the case of analytes initially comprising oligomers, or analytes which oligomerise during the time course of the assay, the plot of ΔR vs. ΔF is also initially linear, but the average gradient is significantly greater than for soluble non-oligomerised proteins, with values of up to about 0.038 Ohm.seconds, so the behaviour of oligomerised analytes can be clearly distinguished from non-oligomerised analytes.

In addition, whilst the ΔR vs. ΔF plot may be initially linear, the inventors have found that for samples comprising oligomer analytes (either initially or formed during the course of the experiment), as the assay proceeds R increases more slowly than F with time, such that the ΔR vs. ΔF plot becomes non-linear. This non-linearity provides another means of distinguishing between oligomerised and non-oligomerised analytes and between analytes with different extents of oligomerisation and can be measured e.g. on a graph or mathematically, or modelled by a computer. For example for a plane curve C, the curvature at a given point P has a magnitude equal to the reciprocal of the radius of an osculating circle (a circle that “kisses” or closely touches the curve at the given point), and is a vector pointing in the direction of that circle's centre. The smaller the radius r of the osculating circle, the larger the magnitude of the curvature (1/r) will be; so that where a curve is “nearly straight”, the curvature will be close to zero, and where the curve undergoes a tight turn, the curvature will be large in magnitude. Hence the curvature, κ, of ΔR=f(ΔF), or ΔR as a function of ΔF can be determined from:

$$κ = \frac{Δ {F}^{″}}{{( 1 + Δ {F}^{′2} )}^{\frac{3}{2}}}$$

where the dash refers to differentiation with respect to ΔR.

This form is widely used in engineering, for example; to derive the equations of bending of beams, deriving approximations to the fluid flow around surfaces (in aeronautics) and the free surface boundary conditions in ocean waves. In some applications, the assumption may be made that the slope is small compared with unity, so that the approximation:

$$κ = \frac{{d}^{2} Δ F}{d Δ {R}^{2}}$$

may be used. This approximation yields a straightforward linear equation describing the phenomenon, which would otherwise remain intractable.

If the curve is defined in polar coordinates as r(θ), its curvature is then:

$$κ ( θ ) = \frac{{r}^{2} + 2 {r}^{′2} - {rr}^{″}}{{( {r}^{2} + {r}^{′2} )}^{\frac{3}{2}}}$$

where here the dash refers to differentiation with respect to θ.

ΔR and ΔF can also be usefully compared by preparing plots of ln(ΔR/ΔF) against time (seconds) and performing vector analysis or Cartesian analysis on the resulting plots. Thus the invention enables distinction between an analyte in oligomerised or non-oligomerised forms, (i.e. the detection of an aggregated analyte against a background of subunits).

Moreover, by comparing the behaviour of a test sample with one or more reference samples or standards having a known degree of oligomerisation, it may be possible to define more precisely the average extent of oligomerisation of the sample.

The invention may also be used to provide information about the kinetics of oligomer formation or de-oligomerisation.

If a sensor surface with a finite number of binding sites is exposed to a sample comprising excess subunit, the system will reach an equilibrium position in which substantially all of the binding sites are occupied by subunit. After reaching this equilibrium position at time tₑ, there will essentially be no further change in F or R, and ΔF and ΔR will reach constant values.

In contrast, if the sensor surface remains in contact with subunits which can oligomerise, then material will continue to accumulate on the sensor surface, due to oligomerisation of the subunit (even though all the specific binding sites on the sensor surface may be occupied), especially if this is catalysed by existing oligomers or other catalyst present in the sample mixture. In such circumstances, it will take longer for the system to reach equilibrium (i.e. tₑ will be much greater) and, in absolute terms the total ΔF and ΔR changes will be greater than for reactions in which only monomer is present. Thus, after an extended period of reaction ΔF and ΔR values will be much further from the origin on ΔR/ΔF plots than for subunit-only systems, and this can be represented graphically or shown by vector analysis. This represents yet another embodiment of the method of the invention.

This type of analysis also leads to a method of analysing the rate of oligomerisation (or de-oligomerisation) of a sample. For example, in the case of a sample with a fixed degree of oligomerisation binding to a sensor, then the value of ln(ΔR/ΔF) should be invariant, or minimally variant with time. However, for the case i) of a sample which is increasing or decreasing in extent of oligomerisation or ii) a sample of subunit that associates with surface-bound oligomer or that catalyses further oligomerisation or iii) for a ligand that induces or prevents process i) or ii) above, then the value of ln (ΔR/ΔF) should vary with time. Analysis of ln(ΔR/ΔF) vs. time gives the observed rate of change of oligomerisation or de-oligomerisation. In the case of an autocatalytic process in which surface-immobilised oligomer induces further oligomerisation of subunits, then this process can continue in time and not reach equilibrium or saturation. Here, a linear fit of the variance of ln(ΔR/ΔF) vs. time can provide the rate of oligomerisation.

In the case in which oligomerisation reaches saturation for whatever reason, or in the case in which the process is reversible (which by definition would lead to an equilibrium position), then there are well known numerical methods for determination of the kinetic constants for such a processes. For example for the simple bimolecular association,

subunit+receptor→subunit−receptor complex,

(in which the receptor may be, for example, a surface-immobilised antibody, surface immobilised monomer or surface immobilised oligomer), the process can be assumed to be a pseudo first order reaction with no interaction between separate receptor molecules. The dissociation rate constant can derived from the equation:

ln(ΔR/ΔF)ₜ=ln(ΔR/ΔF)ₜ_{₀}e^{-k}^{_{d}}^{(t-t}^{₀}⁾

where ln(ΔR/ΔF)ₜ is the response at time t, ln(ΔR/ΔF)ₜ₀ is the response at time t₀ and k_{d} is the dissociation rate constant. The association rate constant can be derived using the equation:

$${ln ( Δ R \text{/} Δ F )}_{t} = \frac{{k}_{a} C {ln ( Δ R \text{/} Δ F )}_{max} ( 1 - {e}^{- ( {k}_{a} C + {k}_{d} ) t} )}{{k}_{a} C + {k}_{d}}$$

where Rₘₐₓ is the maximum response (proportional to the amount of immobilised ligand), C is the concentration of subunit in solution, and kₐ is the association rate constant.

Optionally, or alternatively, it may be useful to measure the angle of the terminal vector (i.e. the vector from the origin to the limiting value of ΔR/ΔF plot at equilibrium on the sensor surface).

Yet another method of analysing the ΔR and ΔF data is to examine the distribution of time points in the ΔR/ΔF plot. For a sample which contains an oligomer or subunit that complexes with surface bound subunit, there will be relatively few time points on the early portion of the plot and a relatively large number of time points on the later part of the plot. Generally, the greater the relative abundance of oligomerised analyte in the sample, the fewer time points will fall on the initially linear portion of the plot.

The invention may, for example, be used to distinguish between the presence, and/or relative amounts of, subunit and oligomeric forms of an analyte. The invention may also be used to screen for molecules (especially non-peptidic small molecules, and peptides containing 1-9 amino acid residues, or small polypeptides containing 10-30 amino acid residues) that can affect (either cause or promote, or prevent or reduce) the oligomerisation of an analyte. Thus, for example, the invention may comprise the step of causing to be present in a reaction mixture a plurality of subunits of an analyte under conditions in which the subunits tend to form oligomers, and a test substance which may affect the oligomerisation of the subunits, and monitoring as aforesaid the extent, if any, of oligomerisation of the subunits.

Alternatively, the inverse approach may be adopted, providing a plurality of subunits under conditions in which oligomerisation tends not to occur, and contacting this mixture with a test substance which may cause or promote oligomersation, and again monitoring as aforesaid the extent, if any, of oligomerisation. Yet another embodiment is to contact a sensor surface with a fully or highly oligomerised sample and conduct a screen for substances which can reverse the oligomerisation (i.e. cause de-oligomerisation). Whichever embodiment is employed, the test compound or substance can be screened over a range of concentrations, to find the IC₅₀ or equivalent (i.e. the concentration of test substance which causes a 50% inhibition of oligomerisation, or 50% de-oligomerisation etc., as appropriate). Those skilled in the art will appreciate that, in such embodiments, the invention provides a method of screening substances to identify those which can promote or inhibit oligomerisation and thus may have potential use as lead compounds for pharmaceutical research or may have actual therapeutic application themselves.

The tendency to form oligomers can be influenced by controlling reaction conditions such as one or more of the following: temperature, pH, ionic balance, presence or absence of enzymes or enzyme inhibitors (such as proteases or protease inhibitors).

As noted above, there are some pathological conditions in which the stage of disease may be associated with the presence and/or relative amount of oligomers, which can be detected or measured by the present invention. Frequently, the relative proportion of oligomerised protein in a clinical sample may be very small (e.g. 1 molecule of oligomer in up to 50,000 normal subunit molecules). The method of the present invention is, however, quite sensitive, since the few molecules of oligomer which may be present have a disproportionately large mass and hence a considerable effect on the ΔF and ΔR values measured.

Nevertheless, it may be desirable to improve the chances of detecting the oligomer molecule by causing relative enrichment of the oligomer in the sample, by concentrating the oligomer and/or reducing the subunits. This can be done, in general terms, by preferentially binding either the oligomer or the subunit and concentrating the former or removing the latter, prior to contacting the sample with the sensor surface. For example, there have been described polymeric molecules (SEPRION™) which preferentially bind aggregated prion proteins (WO 03/073106—Microsens Biotechnologies Limited); or salts of phosphotungstic acid (U.S. Pat. No. 6,916,419—University of California) which have the same property. These molecules are thought to bind the β-pleated sheets which are present in the aggregated form of the protein. Equally there has been disclosed at least one immunoglobulin which binds to an epitope specific for the aggregated prion protein (WO 2005/114214—Prionics). These molecules can be used to concentrate oligomers in the sample, e.g. by affinity chromatography or the like, prior to contacting the sample with the sensor.

These molecules could optionally be coated on the sensor surface (in addition to, or instead of, the enrichment step above), to enhance the selectivity and sensitivity for oligomers from a sample that may contain a mixture of oligomerised and non-oligomerised material.

In general terms, the invention is frequently concerned with the calculation or derivation of relative rather than absolute values. It will often be desirable therefore for the method to include the use of a control sample and/or a control sensor surface. For example, in some embodiments, the method of the invention will comprise the use of the sensor surface in the absence of analyte, in order to characterise the senior surface in such conditions, typically before the sensor surface is contacted with the analyte (but potentially after the surface has been exposed to the sample and any bound analyte subsequently removed). The sensor surface may be exposed to suitable reference liquids to assist in this process e.g. sterile distilled water or buffers, which should preferably be similar in composition to the liquid present in the actual sample, but without the analyte.

Another variation of the general principle of the invention is the possibility of contacting the sample, (preferably the same, aliquot thereof), with a plurality of different sensor surfaces, each having a different loading of binding partner or receptor for the analyte e.g. one surface with a very low loading, one surface with a very high loading, and a third surface with an intermediate loading. Assays of this type can provide additional information about, e.g. the amount of oligomer analyte present in the sample.

Another illustration of a situation in which the present invention might be useful concerns the characterisation of biopharmaceuticals, especially therapeutic antibodies. Typically therapeutic antibodies tend to lose the relevant binding characteristic or other biological activity, and lose solubility, when they become oligomerised. This applies especially to molecules which comprise just a portion of an antibody e.g. F_{ab} or scFv molecules. Thus, the present invention is useful in quality assurance and quality control procedures applied to such biopharmaceuticals.

In a further aspect, the invention provides apparatus suitable for performing the method of the invention. In particular, the apparatus will comprise computer means programmed to receive data input derived from a resonance sensor, and calculate two or more parameters selected from:
series resonance frequency (fₒ), and hence the
series resonance frequency shift (ΔF);
motional resistance (R_{M}), and hence the motional resistance shift ΔR;
motional inductance (L_{M});
motional capacitance (C_{M});
parallel capacitance (C₀);
frequency half-band half-width (Γ);
Q-factor (=f/(2Γ);
dissipative factor (1/Q);
impedance or admittance phase (φ) and hence phase shift (Δφ); and
impedance or admittance amplitude (Z, or Y) and hence amplitude change (ΔZ, or ΔY)
   and further to analyse said calculated parameters to obtain further data which vary according to the extent, if any, of oligomerisation of mass bound to the sensor, and/or its change in time during the course of an assay.

Typically the Apparatus Will Further Comprise One or More Additional Components useful in performing the method. Thus, for example, the apparatus may further comprise any one or more, in any combination, of the following: a plurality of sensors; drive means to cause the sensors to oscillate; receivers to receive signals from the sensors; a flow cell to house a sensor; microfluidics systems to regulate and control the flow of a fluid sample to the sensor; a network analyser and associated circuitry; one or more visual displays to display data; and a printer to provide a printed output.

Preferably the computer means will calculate the resonance frequency shift, ΔF, and/or the motional resistance shift, ΔR, which occur upon binding of analyte to the sensor. Preferably the computer means will calculate both ΔF and ΔR. Most preferably the computer means will calculate both ΔF and ΔR and, in general terms, further analyse the relationship between these values. Preferably the computer means will be programmed accordingly.

In particular the computer means may investigate the relationship between ΔF and ΔR in terms of calculating or deriving the gradient and/or degree of curvature in plots of ΔR against ΔF; and/or determining the terminal values of ΔF and ΔR, and/or analyse the behaviour of (abs(ΔF/ΔR)) against time.

In one embodiment, a network analyser produces scans of admittance vs. frequency several times a second, and the received raw admittance data are processed in firmware by a digital signal processor, which extracts the real and imaginary parts from each frequency sweep, and sends a stream of data comprising sets of (real) Re(Y) and (imaginary) Im(Y) values as a function of frequency during the scans to a PC, via a USB 2 bus interface. The PC runs a programme which calls a subroutine to fit the sets of admittance data to the model, and extract the selected parameters F₀ and R_{M} as a function of the time of the scan. A further routine running on the PC and written in a high level computer language such as C displays the parameters graphically as a function of time. The PC will usually perform some averaging, to reduce the amount of data to be handled, and typically, display plots of, e.g. ΔF(t) and ΔR_{M}(t). Processed parameter data as a function of time are stored in files which can be read by data analysis software to select portions of the data to analyse and determine rate constants and the like from the ΔF(t) data. In this embodiment the PC is also provided with a further subroutine for the display and calculation of parameters appropriate for the measurement of aggregation, for example: ln(abs (ΔF/ΔR)) against time; these data may then be subsequently analysed, for example by fitting to rate equations, or by estimation from sample standards having known extents of aggregation, as described above, to provide user-relevant data. Such data analyses may be incorporated into an assay and sample management function provided as part of the software in the PC.

In an alternative embodiment the Digital Signal Processor may be programmed to do the parameter fitting in real time, and stream the parameters to the PC.

In yet another aspect the invention provides a computer program, and an associated computer device incorporating the program. The program may, optionally, for input F and R_{M} values calculate ΔF and ΔR, and from calculated or input ΔF and ΔR values analyse the data e.g. by producing a plot of ΔF against ΔR etc., and then perform a further calculation (e.g. derive the curvature and/or gradient of a ΔF/ΔR_{M} plot) to obtain and display numerical values which, to the person skilled in the art, will convey information regarding the presence and/or relative amount of oligomeric material in the sample being analysed.

For the avoidance of doubt it is hereby explicitly stated that any feature described herein as “desirable”, “preferred”, “convenient”, “advantageous” or the like may be used in the invention in isolation, or in combination with any one or more other features so described, unless the context dictates otherwise.

Further, the content of all publications cited herein is hereby incorporated by reference.

The invention will now be further exemplified by way of illustrative example and with reference to the accompanying drawings, in which:

FIG. 1A illustrates the Butterworth Van Dyke model for an unloaded crystal resonator;

FIG. 1B illustrates the modified Butterworth Van Dyke (MBVD) model, to represent the behaviour of a loaded crystal resonator;

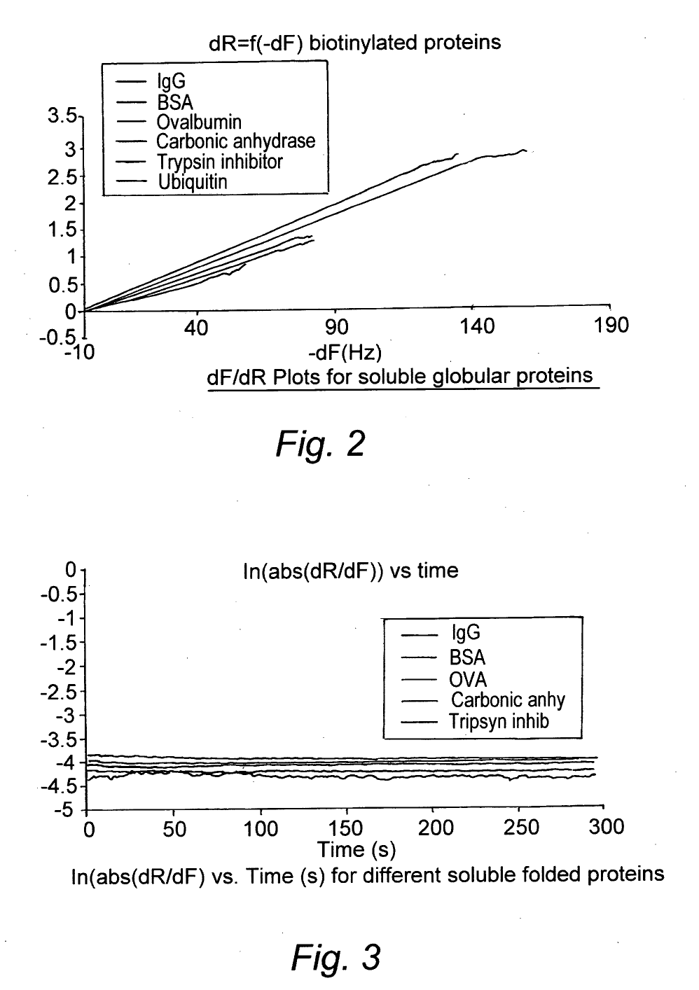

FIG. 2 is a graph of ΔR against OF for binding to a sensor surface of various soluble, non-aggregated proteins of different molecular weights;

FIG. 3 is a graph of ln(absolute (ΔR/ΔF) against time for binding to a sensor surface of various, soluble non-aggregated proteins;

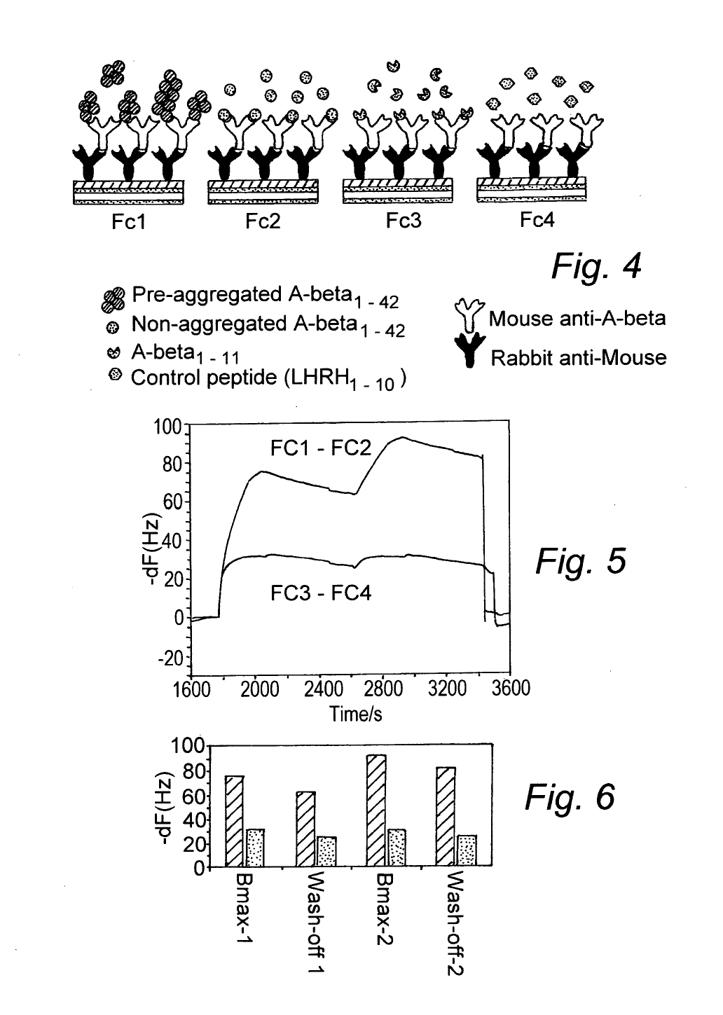

FIG. 4 is a schematic representation of various binding events measured in some of the examples;

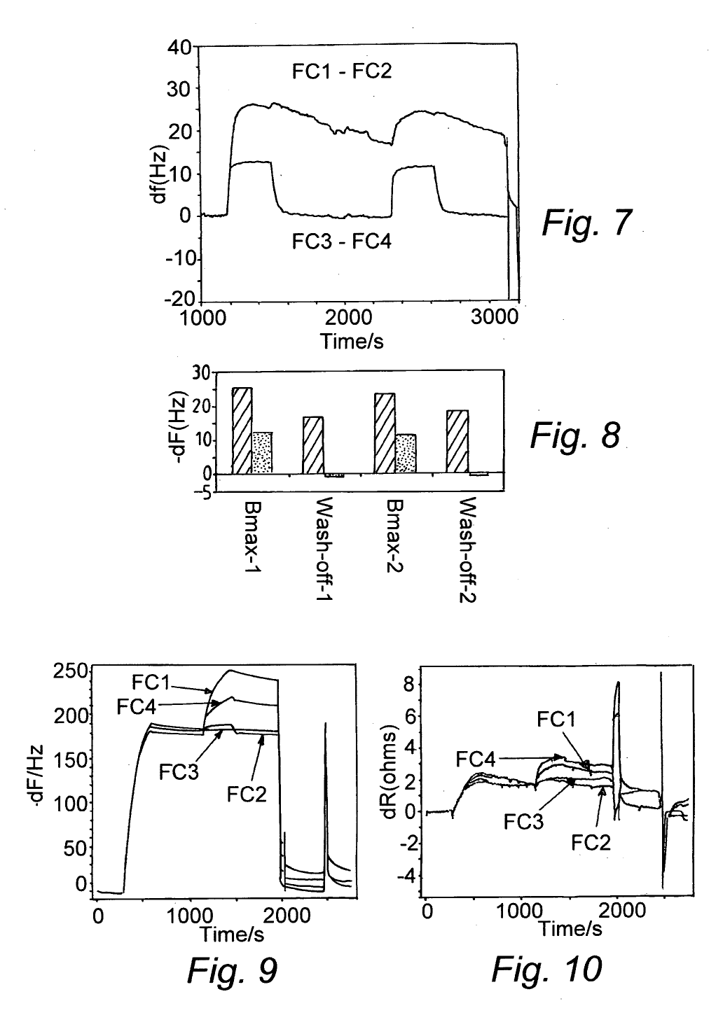

FIGS. 5 and 7 are graphs of ΔF (Hz) against time (seconds);

FIGS. 6 and 8 are bar charts showing ΔF for various experiments;

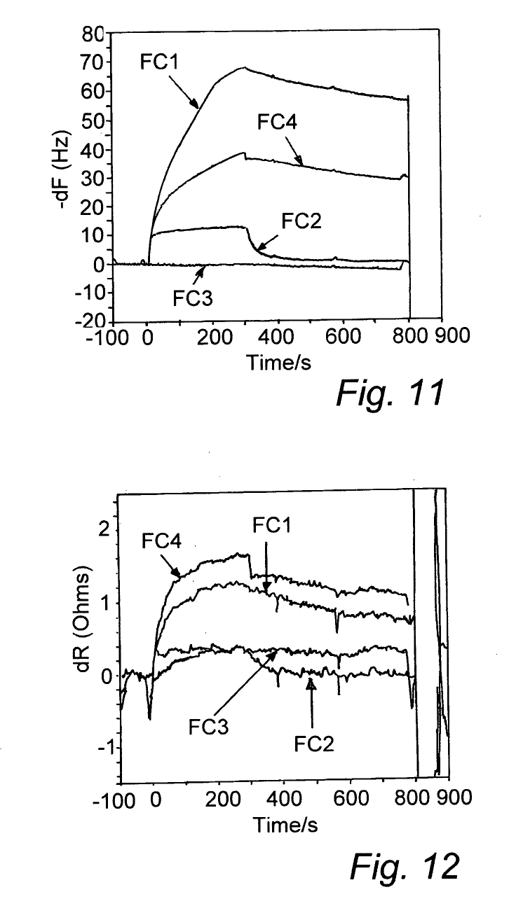

FIGS. 9 and 11 are graphs of ΔF (Hz) against time (seconds);

FIGS. 10 and 12 are graphs of ΔR (Ohms) against time (seconds);

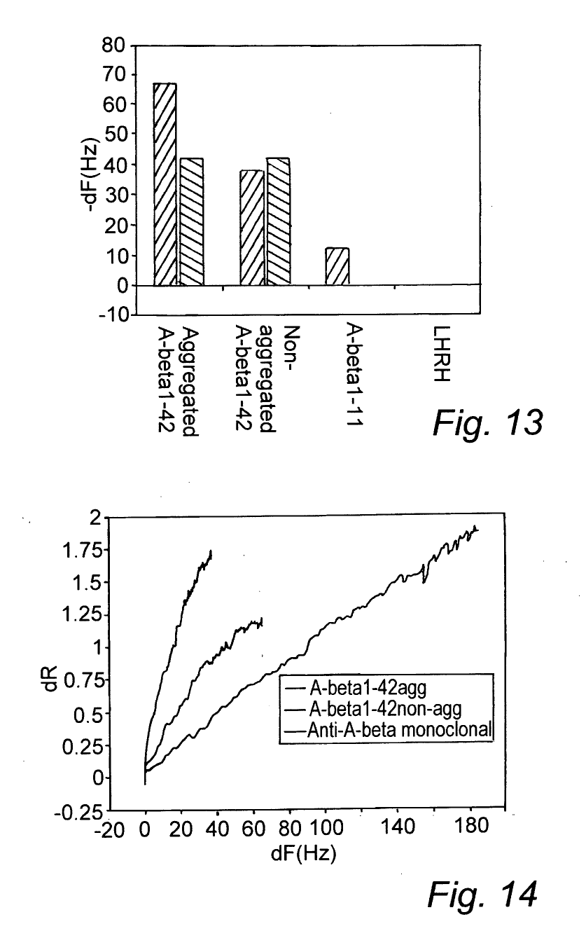

FIG. 13 is a bar chart of ΔF (Hz);

FIG. 14 is a graph of ΔR against ΔF; and

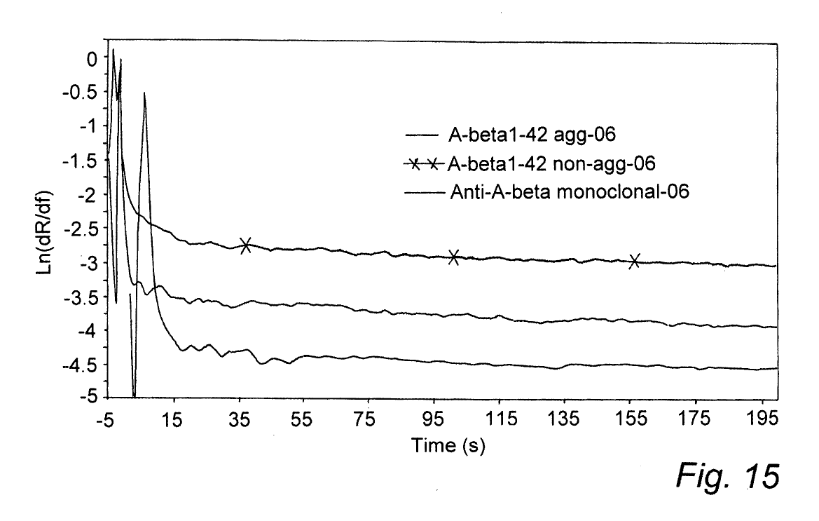

FIG. 15 is a graph of ln(ΔR/ΔF) against time (seconds), for initially oligomerised analyte (solid line); initially non-oligomerised analyte (broken line with crosses) and non-oligomerising analyte (broken line)

### EXAMPLE 1

FIG. 1a represents the Butterworth Van Dyke model for the behaviour of an oscillating sensor, such as a quartz crystal resonator, in a vacuum. The crystal behaves in a manner analogous to a capacitance, an inductance and a resistance, all in series, (denoted C_{q}, L_{q} and R_{q} respectively) with a further capacitance (C₀) in parallel to represent extraneous capacitances (e.g. the electrical connections to the crystal etc.). However, this simple model does not accurately relate to real systems.

FIG. 1b illustrates the modified Butterworth Van Dyke model (MBVD), which is a better representation of behaviour in practice. The sensor has the same properties (C₀, C_{q}, L_{q} and R_{q}) as previously, but additionally there are components L₁ and R₁ (the load inductance and the load resistance respectively). The load resistance reflects the damping effect of, for example, friction between the “loaded” sensor (i.e. the receptor-coated sensor together with analyte molecules bound thereto) and the surrounding fluid medium (in practice usually an aqueous liquid). The load inductance reflects the increased inertia due to the increased mass of the sensor (as a result of the mass of analyte captured on the sensor).

The present invention rests, at least in part, on the principle that the values of L_{L} and R_{L} do not depend solely on the mass of substance captured by the crystal, but also depend on the molecular shape and arrangement of that mass such that, for example, binding of equivalent masses of monomeric and oligomeric material to a sensor can be distinguished.

### EXAMPLE 2

Typical apparatus useful for performing the method of the invention is as follows.

In use, an analyte fluid is passed into a flow cell (e.g. as described in WO 02/12873), and the sensor is operated in the way described below. An associated computer causes a synthesizer to generate a sinusoidal signal at a starting frequency and then progressively to increase the frequency to a given maximum. The range of frequencies spanned is intended to include the resonance frequency of the sensor (and any substance bound onto the receptors immobilised thereon). As the variable frequency signal is fed to the sensor, its admittance is measured by a receiver, and this is stored in the computer as a function of frequency. The admittance can be used to provide a measurement of the resonance frequency of the sensor, and/or the Q factor of the sensor (with the bound substance) in the fluid medium. For example, the frequency at which the admittance is at a maximum will correspond to the resonance frequency of the sensor since it is at this frequency that the sensor will accept the maximum amount of driving energy from the driver.

Preferably the apparatus will also include at least one reference sensor, run in parallel with the test sensor. By this is meant that receptor immobilised on the surface of the reference sensor element has no affinity for the analyte species and thus will not bind any analyte and generate added mass, but in all other respects the reference sensor element, cell and fluid is the same as the test sensor and flow cell. Because the resonance frequency and Q of a quartz crystal sensor element depends on fluid properties as well as attached mass, this enables the influence of the fluid properties to be substantially removed from the response of the sensing elements which carry receptors and are exposed to the same fluid, and a measurement of the amount and character of attached mass to be isolated.

In practice, before measuring the admittance characteristics of the sensor under the analyte containing fluid, the correct frequency scan range will normally be determined. This can be done using an iterative approach: starting with a broad frequency scan of say 500 kHz centred around the normal resonance frequency of the given sensor (i.e. when no substance is bound onto the receptor, and the sensor is in a buffer fluid) data is collected at a number of frequencies. This is fitted against well-known equations to determine rough estimates of the actual resonance frequency and quality factor or resistance and inductance associated with the sensor. The scan range is then narrowed, using this data, and the process is repeated to get improved estimates of resonance frequency, and Q (equivalent to the width of the resonance). By repeating with narrower frequency ranges the estimates approach constant values and this can be used to calculate the optimum sweep range. The resonant frequency can then be determined by fitting the data in this narrow range to a physical model of the resonance. In general resonators will have slightly different resonances and Q factors, and thus different sweep ranges. The range for a particular resonator is stored in the control software for use in the measurement phase.

### EXAMPLE 3

### Biotinylation of Proteins

Protein biotinylation was achieved using Immunoprobe biotinylation kit from Sigma-Aldrich (Catalog No. BK-101). Biotinylation was performed by reacting the protein with the water-soluble reagent BAC-SulfoNHS. Following the reaction, the biotinylated protein was separated from the by-products by a gel filtration column (Sephadex G-25 catalog No. B 4783) using 0.01M Phosphate Buffered Saline (PBS) pH 7.4 as eluent. Consequently the biotinylated protein stock solutions were in that buffer. The ratio of biotin to protein was determined by the avidin-HABA assay. The absorption of the avidin-HABA complex at 500 nm decreased proportionally with increasing concentration of biotin as the HABA dye was displaced from avidin due to the higher affinity of avidin for biotin.

Molar ratios of BAC-SulfoNHS to protein of between 3:1 and 5:1 were used to obtain a biotin to protein ratio around 1.

For consistency, the non-biotinylated proteins used as controls were prepared in the same buffer at the same concentration. Aliquots of all the biotinylated proteins stocks were stored at −20° C. For the binding assays, 1 μM protein solutions were prepared by diluting the protein stock solutions with PBS running buffer (Pre-made PBS Sigma cat no. D8537).

TABLE 1
Summary of biotinylated proteins used.

| Biotinylated | Molecular Weight | | Concentration |
|---|---|---|---|
| proteins | (Da) | Biotin/protein | (nmol/ml) |
| Immunoglobulin G | 150000 | 1.2 | 25 |
| BSA | 66400 | 1.1 | 70 |
| Ovalbumin | 45000 | 0.8 | 59 |
| Carbonic Anhydrase | 30000 | 0.8 | 88 |
| Trypsin Inhibitor | 20100 | 1.2 | 225 |
| Ubiquitin | 8500 | 1.0 | 343 |
| Insulin chain B | 3500 | 0.9 | 129 |

Immobilisation of Mouse Anti-Biotin Antibody onto the Sensor Surface.

Mouse anti-biotin antibodies (Jackson Immunologicals) were covalently attached to the surface of a quartz crystal resonance sensor by amine coupling. The carboxyl groups on the surface of an Akubio LINKit™ Covalent A cassette were activated using EDC/NHS chemistry (3 minute injection at flowrate 25 μL/min), after which 50 μg/ml of the Mouse anti-biotin antibody solution in acetate buffer pH 5.5 was injected (3 minute injection at flowrate 25 μL/min) The surface was finally capped with 1M Ethanolamine (3 minute injection at flow rate 25 μL/min).

### D-Biotin Binding.

A 10 μM solution of d-biotin (Sigma) in running buffer was injected (1 minute injection at flow rate 25 μL/min) after immobilization of the antibodies to serve as an internal reference allowing the assessment of the antibody activity.

### Binding of Biotinylated Proteins.

1 μM solutions of biotinylated proteins and non-biotinylated homologues (as controls) were prepared by diluting the stock solutions prepared above with PBS running buffer. The protein solutions were injected (5 minute injection at flowrate 25 μL/min) over the sensor surfaces. Biotinylated protein was injected in the first channel and the non-biotinylated analog as a control was injected in the second channel. At least three repeats of the binding assay were performed.

ΔF and ΔR data were extracted using Akubio RAPid™ instruments and software, by fitting the imaginary part of the admittance data as a function of frequency, at each point in time during the course of the assay, to the equation;

$$Im ( Y ) = \frac{Q}{R} \frac{Ω}{( 1 + {Q}^{2} {Ω}^{2} )} + ω {C}_{0} ;$$

$$where$$

$$F = ω \text{/} π , \\ and$$

$$Ω = \frac{ω}{{ω}_{0}} - \frac{{ω}_{0}}{ω}$$

The fitting procedure extracted 4 parameters; F₀, Q, R_{M} and C₀. As previously described, ΔR corresponds to the change of the fitted value of R_{M} as the binding occurs.

The results for the various proteins are shown in FIG. 2, which is a plot of ΔR against ΔF. The graph shows that the plots for each protein are essentially linear, and the gradients of the plots are broadly similar. The data are presented in a different form in FIG. 3, which is a graph of ln(abs(ΔR/ΔF)) against time in seconds. Following the initial noise the plots become straight lines with essentially no gradient, showing that ΔR/ΔF is a constant ratio for the binding of these proteins.

### EXAMPLE 4

### Misfolded/Aggregated Analyte

Rabbit anti-mouse was covalently coupled to all the sensor surfaces, and mouse anti-A-beta monoclonal antibody (5 μg/ml, 0.33 nM) was captured onto all 4 surfaces. A-beta and control peptides were injected across individual surfaces, as shown schematically in FIG. 4. The numbers (1-42), 1-11 etc. refer to the number of amino acid residues in the peptide)

### Reagents:

1) Deionised H₂O sterile-filtered & degassed.
2) Running buffer PBS
3) EDC 0.4M
4) NHS 0.1 M
5) Coupling buffer: 10 mM Na-Acetate pH 5.5
6) Ethanolamine 1M pH 8.5 (all coupling reagents from Coupling kit, expiry February 2007
7) Rabbit anti Mouse IgG (Jackson 315-005-008 lot: 64640, 2.4 mg/ml)
8) 6E10 mouse monoclonal anti-A-beta, 1 mg/ml, 5 μl aliquots obtained from BioQuote-Signet
9) Mouse anti-biotin (Jackson, lot 64880, 1.3 mg/ml) was obtained from Stratech.
10) A-beta1-42 0.5 mg aliquots of lyophilised powder obtained from American Peptide Company and re-purified as described in Kokkoni, N., Stott, K., Amijee, H., Mason, J. M. and Doig, A. J. (2006). Biochemistry 45 (32), 9906-18
11) A-beta1-11 2.2 mg aliquot of lyophilised powder obtained from American Peptide Company
12) Gly10-OH-Luteinizing Hormone Releasing Hormone (LHRH), Sigma Cat No. L-8008, lot 123K1472, MWt1183.3, stock prepared at 5 mg/ml in MilliQ Water by KT on Feb. 7, 2004
13) Regeneration solution 1: 100 mM HCl
14) Regeneration solution 2: 20 mM NaOH

### Experimental Protocols

Immobilisation of ligands: Rabbit anti-mouse (Jackson) lot number 64640, prepared at 50 μ/ml, 20.8 μl of 2.4 mg/ml stock in 1 ml 10 mM acetate buffer, pH 5.5.
Immobilisation Assay: EDC-NHS, 3 mins, 25 μl/min Fc1-4 (post-injection=0). RaM, 3 mins, 25 μl/min, Fc1-4 (post-injection=0) Ethanolamine, 3 mins, 25 μl/min, Fc1-4 (post-injection=0)
Surface stabilization/regeneration: 3× (60 sec 100 mM HCl, 30 sec 20 mM NaOH)
Buffer changeover: PBS+1% DMSO prepared using 400 ml of the same degassed PBS stock used for immobilisation+4 ml DMSO.

Preparation of A-beta 1-42 samples: Protein as lyophilised powder is taken up from solvent solution into buffer according to stage 10) above, at which point aggregation is initiated. Assay measurements were started within approximately 10 minutes of the buffer solution preparation, at which time the resulting mixture comprises mostly monomer plus a small amount of early stage aggregated protein.

### Peptide Comparison 1: Mouse Anti-A-Beta Monoclonal Antibody Capture, Binding of Pre-Aggregated and Non-Aggregated A-Beta1-42

1) Mouse anti-biotin, lot 64880, 1.3 mg/ml, 3.85 μl made up to 5 μg/ml in PBS+1% DMSO
2) Mouse anti-A-beta (6E10), 1.0 mg/ml, 5 μl made up to 5 μg/ml in PBS+1% DMSO
3) A-beta1-42 (pre-aggregated), prepared at 250 μg/ml in PBS on Aug. 24, 2006 and stored at −80° C., thawed and diluted to 1 μg/ml by adding 8 μl to 2 mls PBS+1% DMSO, allowed to stand at room temperature throughout preparation period, and during loading into the instrument manifold.
4) A-beta1-42 (non-aggregated), prepared at 1 mg/ml in DMSO (0.5 mg aliquot diluted with 500 μl DMSO) on the day of use, then diluted to 100 μg/ml by adding 100 μl to 900 μl DMSO. Fresh aliquots prepared immediately before Injections 2 and 3 described below.
5) 100 μg/ml stock in 100% DMSO diluted 10 μl stock+1 ml PBS immediately before sample pickup by the instrument needle, to give 1 μg/ml in PBS+1% DMSO.
6) Perform antibody capture and A-beta1-42 binding assay according to the following Summary:
   Injection 1:
   a. Fc1+3: Anti-A-beta (6E10) 5 μl/ml, 25 μl/min, 5 mins, (post-injection=0)
      Fc2+4: Anti-biotin 5 μg/ml, 25 μl/min, 5 mins, (post-injection=0)
   Injection 2:
   b. Fc1+2: Pre-aggregated A-beta1-42 1 μg/ml, 25 μl/min, 5 mins, (post-injection=60)
      Fc3+4: Non-aggregated A-beta1-42 1 μg/ml, 25 μl/min, 5 mins, (post-injection=60)
   Injection 3:
   c. Fc1+3: Pre-aggregated A-beta1-42 1 μg/ml, 25 μl/min, 5 mins, (post-injection=60)
      Fc2+4: Non-aggregated A-beta1-42 1 μg/ml, 25 μl/min, 5 mins, (post-injection=60)
   Injection 4:
   d. Fc1 to 4: 100 mM HCl, 25 μl/min, 60 sec, (post-injection=0)
   Injection 5:
   e. Fc1 to 4: 20 mM NaOH, 25 μl/min, 30 sec, (post-injection=0)

### Peptide Comparison 2: Mouse Anti-A-Beta Monoclonal Antibody Capture, Binding of Non-Aggregated A-Beta1-42 and A-Beta1-11

7) Mouse anti-biotin, lot 64880, 1.3 mg/ml, 3.85 μl made up to 5 μg/ml in PBS+1% DMSO
8) Mouse anti-A-beta (6E10), 1.0 mg/ml, 5 μl made up to 5 μg/ml in PBS+1% DMSO
9) A-beta1-11 (prepared at 2 mg/ml in PBS on Feb. 10, 2006 and stored at −80° C.), thawed and diluted to 200 μg/ml in PBS+1% DMSO, diluted to 1 μg/ml by adding 10 μl to 2 mls PBS+1% DMSO
10) A-beta1-42 (non-aggregated), prepared as described above.
11) Perform antibody capture and A-beta1-42 binding assay according to the following Summary:
   Injection 1:
   a. Fc1+3: Anti-A-beta (6E10) 5 μg/ml, 25 μl/min, 5 mins, (post-injection=0)
      Fc2+4: Anti-biotin 5 μg/ml, 25 μl/min, 5 mins, (post-injection=0)
   Injection 2:
   b. Fc1+2: Non-aggregated A-beta1-11 1 μg/ml, 25 μl/min, 5 mins, (post-injection=60)
      Fc3+4: Non-aggregated A-beta1-42 1 μg/ml, 25 μl/min, 5 mins, (post-injection=60)
   Injection 3:
   c. Fc1+3: Non-aggregated A-beta1-11 1 μg/ml, 25 μl/min, 5 mins, (post-injection=60)
      Fc2+4: Non-aggregated A-beta1-42 1 μg/ml, 25 μl/min, 5 mins, (post-injection=60)
   Injection 4:
   d. Fc1 to 4: 100 mM HCl, 25 μl/min, 60 sec, (post-injection=0)
   Injection 5:
   e. Fc1 to 4: 20 mM NaOH, 25 μl/min, 30 sec, (post-injection=0)
      Peptide Comparison 5: A Repeat of Peptide Comparison 3, but with Freshly Prepared Reagents
1) Mouse anti-A-beta monoclonal antibody (6E10), 1.0 mg/ml, 5 μl made up to 5 μg/ml in PBS+1% DMSO
2) Pre-aggregated A-beta1-42 prepared as described above—but using freshly prepared solution.
3) Non-aggregated A-beta1-11 prepared as described above—same solution used
4) Non-aggregated A-beta1-42 prepared as described above—freshly prepared
5) LHRH, stock prepared at 5 mg/ml in MilliQ Water, diluted to 100 μg/ml in PBS+1% DMSO (10 μl in 500), diluted to 1 μg/ml in PBS+1% DMSO (10 μl in 1 ml)
6) Perform antibody capture and peptide binding assay according to the following Summary:
   Injection 1:
      Fc1 to 4: Anti-A-beta (6E10) monoclonal 5 μg/ml, 25 μl/min, 5 mins, (post-injection=0)
   Injection 2:
      Fc1: A-beta1-42 (non-aggregated) 1 μg/ml, 25 μl/min, 5 mins, (post-injection=60)
      Fc2: A-beta1-11 (non-aggregating), 1 μg/ml, 25 μl/min, 5 mins, (post-injection=60)
      Fc3: A-beta1-42 (non-aggregated) 1 μg/ml, 25 μl/min, 5 mins, (post-injection=60)
      Fc4: LHRH1-10 (non-aggregating) 1 μg/ml, 25 μl/min, 5 mins, (post-injection=60)
   Injection 3:
      Fc1 to 4: 100 mM HCl, 25 μl/min, 60 sec, (post-injection=0)
   Injection 4:
      Fc1 to 4: 20 mM NaOH, 25 μl/min, 30 sec, (post-injection=0)

Rabbit anti-mouse immobilization levels of between 760-800 Hz were obtained.

This protocol injection 2 was used to obtain the aggregated and non-aggregated data of FIGS. 7,8,9,10; The anti A-beta monoclonal antibody data were taken from this protocol of injection 1.

### Results

### 6E10 Anti-A-Beta Capture and Peptide Binding Comparison 1

### (Pre-Aggregated vs Non-Aggregated A-Beta1-42)

Aligned and control-subtracted A-beta binding responses only are shown in FIG. 5. Upper trace is Fc1 (aggregated A-beta1-42, 1 μg/ml, 0.22 μM)-Fc2 (aggregated A-beta1-42 control); lower trace is for Fc3 (non-aggregated A-beta1-42, 1 μg/ml, 0.22 μM)-Fc4 (non-aggregated A-beta1-42 control). Aggregated and non-aggregated A-beta maximal binding levels, and levels at the end of the washout after injection are plotted as a histogram in FIG. 6. Pairs of columns are FC1-FC2 (left) and FC3-FC4 (right).

These data show that pre-aggregated A-beta1-42 binds to a higher level than the same concentration of non-aggregated A-beta1-42, and that subsequent additions of Pre-aggregated A-beta1-42 produce a larger response. In contrast, non-aggregated A-beta1-42 produces a smaller response, and repeated injections on this timescale do not produce an additive response.

The saturation of the non-aggregated A-beta monomers shows the finite binding capacity of the surface for this molecule, whereas for the aggregated form, despite a similar binding capacity, the response is larger, therefore the unit mass binding to the surface binding sites must be larger. i.e. it is comprised of more than monomers. Furthermore the fact that subsequent additions of aggregated 1-42 do not lead to saturation of the surface, implies that further aggregation between surface-captured and soluble analyte is occurring.

### 6E10 Anti-A-Beta Capture and Peptide Binding Comparison 2

### (Non-Aggregated A-Beta1-42 vs A-Beta1-11)

Aligned and controlled subtracted A-beta binding responses only are displayed in FIG. 7 The upper trace is for Fc1 (non-aggregated A-beta1-11, 1 μg/ml, 0.75 μM)-Fc2 (non-aggregated A-beta1-11 control), the lower trace is that for Fc3 (non-aggregated A-beta1-42, 1 μg/ml, 0.22 μM)-Fc4 (non-aggregated A-beta1-42 control). Non-aggregated A-beta1-42 and 1-11 maximal binding levels, and levels at the end of the washout after injection are plotted as a histogram in FIG. 8 (FC1-FC2 (left) and FC3-FC4 (right)).

These data show that non-aggregated A-beta1-11 binds and rapidly completely dissociates from the surface; and that a second injection achieves the same binding level as the first. This indicates that the binding capacity of the surface has reached its equilibrium level. Non-aggregated A-beta1-42 binds more slowly, produces a larger response as expected from its greater mass, and dissociates more slowly. Similarly, repeated injections do not produce an additive response.

### 6E10 Anti-A-Beta Monoclonal Antibody Capture and Peptide Binding Comparison 5

(Pre-Aggregated and Non-Aggregated A-Beta1-42, A-Beta1-11 and LHRH Control, at 1 μg/ml)

Raw data are displayed in FIGS. 9 and 10. Fc1=(aggregated A-beta1-42), Fc2=(non-aggregated A-beta1-11), Fc3=(LHRH control peptide), Fc4=(non-aggregated A-beta1-42). FIG. 9 plots ΔF (in H_{z}) against time (in seconds) and FIG. 10 plots ΔR (Ohms) against time (in seconds). The capture of the anti-A-beta monoclonal antibody is the first rising phase, and the binding of the analytes is the second rising phase.

Aligned and controlled subtracted A-beta binding responses for ΔF and ΔR are shown in FIGS. 11 and 12. Fc1=(aggregated A-beta1-42), Fc2=(non-aggregated A-beta1-11), Fc3=(LHRH control peptide), Fc4=(non-aggregated A-beta1-42)

These data show that aggregated A-beta1-42 binds as rapidly as non-aggregated A-beta1-42, though it produces a larger binding response, and that A-beta1-11 rapidly binds and dissociates, while LHRH, as a control peptide, does not bind at all.

Comparison of the maximal binding responses achieved (same bars as for the traces above), and prediction of the non-aggregated and pre-aggregated A-beta1-42 binding response amplitudes based on a molecular weight scaling factor for the A-beta1-11 binding response (grey bars) are shown in FIG. 13.

From the data obtained in these experiments, a plot of ΔR against ΔF was prepared, for the binding of initially aggregated Abeta 1-42, aggregatable Abeta 1-42, and a non-aggregating molecule (Anti-Abeta monoclonal) for comparison, shown in FIG. 14. It is apparent from this figure that binding of the non-aggregating protein results in a substantially linear plot. The initially non-aggregated Abeta 1-42 gives an initially linear plot but with a steeper gradient. As more aggregate forms the plot deviates from linearity. The aggregated Abeta 1-42 plot has the steepest gradient. Both the latter plots are clearly distinguishable from the monoclonal antibody-binding plot. FIG. 15 shows the data presented as a plot of ln (ΔR/ΔF), with clearly distinguishable results for the aggregates and (globular) monoclonal antibody.

1-29. (canceled)

30. A method of detecting the presence and/or amount of an oligomer analyte in a liquid sample, the method comprising the steps of:
(a) contacting a sample comprising the oligomer with an oscillating sensor surface, which surface is provided with a receptor that binds directly or indirectly to at least one component of the oligomer, so as to cause direct or indirect binding of the oligomer to the surface;
(b) using a detection circuit to measure or calculate at least two of the following parameters:
series resonance frequency (fₒ), and hence the series resonance frequency shift (ΔF);
motional resistance (R_{M}), and hence the motional resistance shift ΔR;
motional inductance (L_{M});
motional capacitance (C_{M});
parallel capacitance (C₀);
frequency half-band half-width (Γ);
Q-factor (=f/(2Γ);
dissipative factor (1/Q);
impedance or admittance phase (φ) and hence phase shift (Δφ); and
impedance or admittance amplitude (Z, or Y) and hence amplitude change (ΔZ, or ΔY);
(c) and analysing the calculated values to derive data which vary according to the presence and/or amount of oligomer analyte in the sample;
(d) and repeating the measurements continuously or intermittently to derive data which vary according to the presence and/or amount of oligomer analyte in the sample to be calculated as a function of elapsed time.

31. A method according to claim 30, wherein the sensor comprises a quartz crystal resonator and is selected from the group consisting of:
piezo-electric devices, surface transverse shear mode (TSM) devices, surface acoustic wave (SAW) devices, acoustic plate mode devices, flexural plate wave devices, magnetic acoustic devices, atomic force microscopy devices, micro-electromechanical devices, tuning forks, and derivatised membranes.

32. A method according to claim 30, wherein at least part of the sensor surface is coated with an electrically conducting substance.

33. A method according to claim 30, wherein the receptor molecule is selected from the group consisting of: an antibody or an antigen-binding fragment or variant of an antibody; an antigen; a peptide; a single-stranded oligo- or polynucleotide; a molecule containing at least a ligand-binding portion of a eukaryotic cell surface receptor; a lectin; a polysaccharide; an aptamer; and an affibody.

34. A method according to claim 30, comprising calculation or derivation of fₒ, ΔF, R_{M} or ΔR, wherein the analysing step comprises plotting ΔF against ΔR (or vice versa) or plotting Ln (ΔR/ΔF) against time and calculating the degree of curvature and/or the gradient of the plot.

35. A method according to claim 30, wherein the oligomer analyte comprises a molecule of pathological significance.

36. A method according to claim 35, wherein the analyte comprises an oligomer of a molecule selected from the group consisting of: β-amyloid protein; β-amyloid peptides, especially βA4 peptide; alpha-synuclein; huntingtin; and prion protein.

37. A method according to claim 30, performance of which allows determination of the relative amount or extent of oligomerisation in the sample.

38. A method according to claim 30, wherein the analyte becomes increasingly oligomerised during performance of the method, and the analysing step allows determination of the rate of oligomerisation.

39. A method according to claim 30, wherein the analyte is initially in a fully or highly oligomerised state and becomes less oligomerised during performance of the method, and the analysing step allows determination of the rate of deoligomerisation.

40. A method according to claim 30, wherein the sample is contacted with a test substance before and/or during the step of contacting the sample with the sensor, and noting the effect, if any, of the test substance on the rate or extent of aggregation or oligomerisation of the analyte.

41. A method of screening a test substance for the ability to affect the oligomerisation of an analyte, the method comprising the step of contacting the test substance with a sample comprising the analyte, and performing a method in accordance with claim 30 and determining the presence, amount or rate of formation of oligomerised or deoligomerised analyte.

42. A method according to claim 30, wherein the sample comprises a mixture of both oligomer analyte and monomers thereof.

43. A method according to claim 42, wherein the monomer is present in greater concentration than the oligomer.

44. A method according to claim 30, further comprising the steps of calculating the absolute or relative amount of subunit or monomer in the sample, and calculating the oligomer:subunit or oligomer:monomer ratio.

45. Apparatus for performing the method of claim 30, the apparatus comprising:
an oscillating sensor surface, which surface is provided with a receptor that binds directly or indirectly to at least one component of the oligomer, so as to cause direct or indirect binding of the oligomer to the surface; and
computer means programmed to receive data input derived from a resonance sensor, and calculate two or more parameters selected from:
series resonance frequency (fₒ), and hence the
series resonance frequency shift (ΔF);
motional resistance (R_{M}), and hence the motional resistance sift ΔR;
motional inductance (L_{M});
motional capacitance (C_{M});
parallel capacitance (C₀);
frequency half-band half-width (Γ);
Q-factor (=f/(2Γ);
dissipative factor (1/Q);
impedance or admittance phase (φ) and hence phase shift (Δφ); and
impedance or admittance amplitude (Z, or Y) and hence amplitude change (ΔZ, or ΔY)
and computer means further to analyse said calculated parameters to obtain further data which vary according to the extent, if any, of oligomerisation of analyte bound to the sensor, and/or its change in time during the course of an assay; wherein the measurements are repeated continuously or intermittently to derive data which can vary according to the amount of oligomer analyte in the sample to be calculated as a function of elapsed time.

46. Apparatus according to claim 45, further comprising any one or more, in any combination, of the following: a plurality of sensors; drive means to cause the sensors to oscillate; receivers to receive signals from the sensors; a flow cell to house a sensor; microfluidics systems to regulate and control the flow of a fluid sample to the sensor; a network analyser and associated circuitry; one or more visual displays to display data; and a printer to provide a printed output.

47. Apparatus according to claim 45, wherein the computer means calculates the resonance frequency shift, ΔF, and/or the motional resistance shift, ΔR, which occur upon binding of analyte to the sensor.

48. Apparatus according to claim 45, wherein the computer means is programmed to analyse the relationship between ΔF and ΔR in terms of calculating or deriving the gradient and/or degree of curvature in plots of ΔR against ΔF; and/or determining the terminal values of ΔF and ΔR; and/or analyse the behaviour of ln(abs(ΔF/ΔR)) against time.
